(19) United States
(12) Patent Application Publication
(10) Pub. No.: US 2022/0361462 A1
(43) Pub. Date: Nov. 17, 2022

(54) ANIMAL MODEL FOR NEURODEGENERATIVE DISORDERS

(71) Applicant: NEURO-BIO LTD, Abingdon, Oxfordshire (GB)

(72) Inventor: Susan GREENFIELD, Abingdon, Oxfordshire (GB)

(21) Appl. No.: 17/642,938

(22) PCT Filed: Sep. 18, 2020
(86) PCT No.: PCT/GB2020/052265

§ 371 Date: Mar. 14, 2022

(30) Foreign Application Priority Data
Sep. 18, 2019 (GB) 1913441.0

(51) Int. Cl. A01K 67/027; A61K 49/00

(52) U.S. Cl. CPC A01K 67/027; A61K 49/0008; A01K 2207/10; A01K 2267/0312; A01K 2227/106; A01K 2227/105

(57) ABSTRACT

The invention relates to animal models, and in particular to novel in vivo animal models for neurodegenerative disorders, such as Alzheimer's disease, Parkinson's disease or Motor Neurone Disease. The invention extends to methods for providing such models. The invention also provides animal models per se and methods for investigating the underlying mechanisms occurring in such neurodegenerative disorders, in particular, Alzheimer's disease, and also extends to models, methods and assays for testing pharmacological test compounds, which may modulate neurological processes, and for drug screening for use in treating neurodegenerative diseases.

The invention relates to animal models, and in particular to novel in vivo animal models for neurodegenerative disorders, such as Alzheimer's disease, Parkinson's disease or Motor Neurone Disease, and to methods for providing such models. The invention provides animal models per se and methods for investigating the underlying mechanisms occurring in such neurodegenerative disorders, in particular, Alzheimer's disease, and extends to models, methods and assays for testing pharmacological test compounds, which may modulate neurological processes, and for drug screening for use in treating neurodegenerative diseases.

Alzheimer's disease (AD) is the most common form of dementia, but the primary events promoting this disorder remain still unravelled. The most popular “amyloid hypothesis” is now being increasingly challenged and so an alternative theory compatible with all clinical features is needed. One such different approach focuses on the distinguishing properties of the neurons that are selectively and primarily vulnerable in AD. They constitute a continuous hub of adjacent cell groups, extending from the basal forebrain (BF) to midbrain and brainstem, which send projections to several brain areas, such as the cortex, hippocampus and olfactory bulb. Despite a heterogeneity of transmitters within this core of susceptible cells, an interesting common feature is that they contain the enzyme, acetylcholinesterase, now established to exert a non-cholinergic function. This non-enzymatic role modulates calcium ion influx into neurons and, hence, it can be trophic or toxic, depending on the dose, availability and neuronal age.

Acetylcholinesterase (AChE) is expressed at different stages of development in various forms, all of which have identical enzymatic activity, but which have very different molecular compositions. The ‘tailed’ (T-AChE) is expressed at synapses and the inventors have previously identified two peptides that could be cleaved from the C-terminus, one referred to as “T14” (a 14-mer peptide), within the other which is known as “T30” (a 30-mer peptide), and which both have strong sequence homology to the comparable region of β-amyloid. The AChE C-terminal peptide “T14” has been identified as being the salient part of the AChE molecule responsible for its range of non-hydrolytic actions. The synthetic 14 amino acids peptide analogue (i.e. “T14”), and subsequently the larger, more stable, and more potent amino acid sequence in which it is embedded (i.e. “T30”) display actions comparable to those reported for ‘non-cholinergic’AChE, where the inert residue within the T30 sequence (i.e. “T15”) is without effect.

Currently, there is no widely accepted in vivo animal model which reproduces the full pathological profile of a neurodegenerative disorder, such as Alzheimer's disease (AD), since the basic mechanisms of neurodegeneration are still poorly understood. Not only do current systems fail to replicate the full clinical profile of the disease, but the majority of those available rely on transgenic animals to reflect a disease where only a small percentage of cases have a clear genetic basis. Moreover, transgenic animals are very expensive to produce and entail lengthy waiting periods for the impairment to become apparent. There is, therefore, an urgent need for an improved animal model or assay, which enables the accurate study of neurodegenerative disorders.

The inventors have developed a hypothesis which they believe accounts for the aberrant processes characterizing Alzheimer's disease, based on the interaction between the α7 nicotinic acetylcholine receptor (α7-nAChR) and the toxic 30-mer peptide, which is cleaved from the acetylcholinesterase (AChE) C-terminus, i.e. T30. Based on this hypothesis, they have created a novel, non-transgenic approach using an in vivo (i.e. a rodent) model, which can be used to study neurodegenerative disorders in a much more physiological scenario than cell cultures.

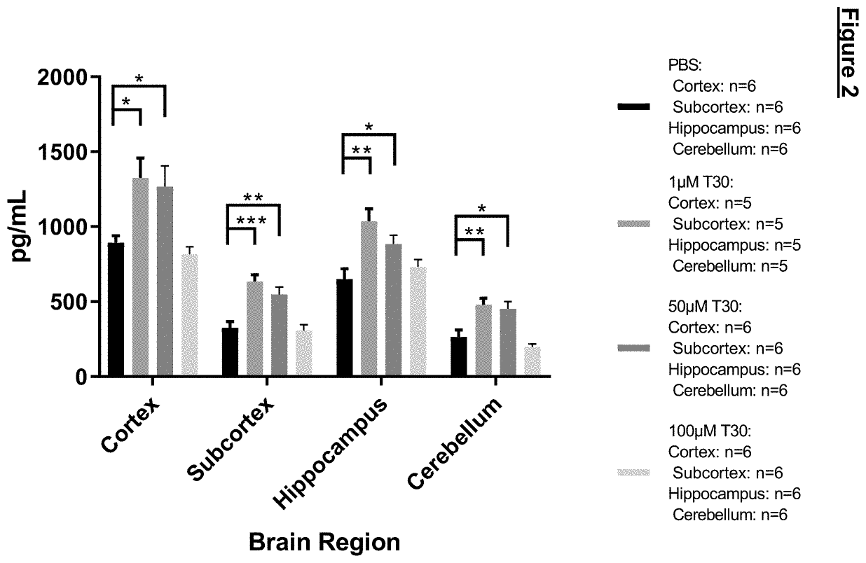

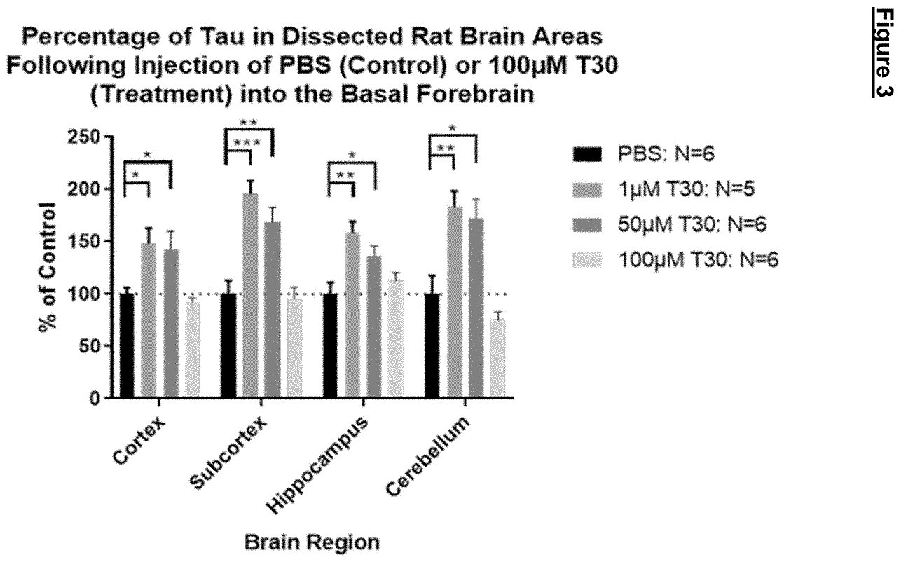

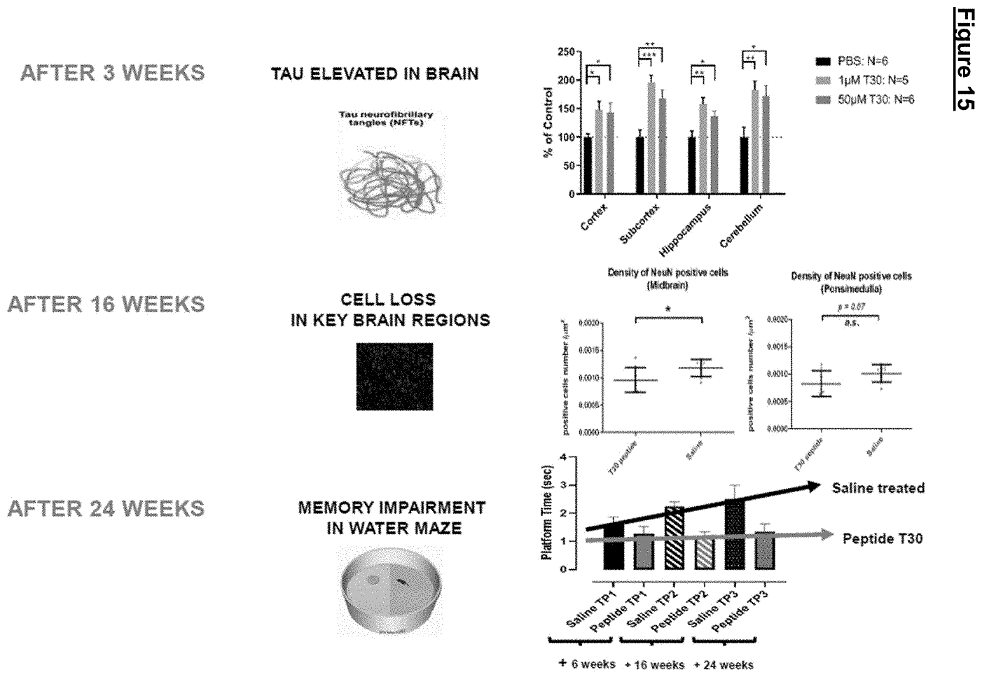

The inventors therefore administered a single dose of the peptide T30 into the medial septum/basal forebrain of a rat, and investigated the T30-mediated modifications on the toxic peptide (T14), as well as on two Alzheimer's disease hallmarks (Tau and Aβ) in four different sections of the brain, namely the cortex, subcortex, hippocampus and cerebellum. In addition, they also analysed the basal forebrain and pons/medulla regions of the brain using immunohistochemistry with quantitative analysis using antibodies. The overall aim was firstly to establish if a single dose of T30 could induce, neurochemically, an ‘Alzheimer's-like’ profile, which is defined as statistically significant increases in AD-related proteins in treatment groups compared to a control, and secondly, to establish at which concentration T30 caused these changes. The ELISA results shown in FIGS. 2 and 3 surprisingly show that the total Tau levels were increased in all four brain regions (cortex, subcortex, hippocampus and cerebellum) upon the T30-peptide administration. Tau is well-known to be the primary pathological marker of Alzheimer's disease, and so the methodology described herein clearly demonstrates the action of T30 in triggering an Alzheimer's disease-like profile in four brain regions. Furthermore, as shown in FIG. 15, a significant decrease in the density of NeuN positive (i.e. NeuN expressing) cells, which are a marker of mature neurons, was observed in the midbrain of rats following administration of the T30 peptide compared to saline-treated animals. In addition, FIG. 15 also shows a deterioration in behaviour of rats treated with T30 using the Morris Water Maze test.

When the data are considered in totality, the inventors firmly believe that this is the first evidence of a toxin (i.e. the T30 peptide) triggering a consistent Alzheimer's-like biochemical profile in the brains of otherwise normal, wild-type rodents. The method described herein suggests a highly novel in vivo approach for monitoring and manipulating neurochemical phenomena contributing to neurodegeneration, in a time-dependant and site-specific manner. This new approach clearly allows the exploration of the early stages occurring during neurodegeneration in a physiological context, maintaining the local neuronal circuitry of the studied region and giving the possibility to monitor its acute response. The application of this methodology could be used to examine many molecular processes, test pharmacological compounds which may regulate these processes and provide a reliable tool for drug screening.

Thus, in a first aspect of the invention, there is provided a method of providing an animal model for a neurodegenerative disease, the method comprising introducing, into the brain of a non-human animal, a peptide comprising or consisting of the amino acid sequence represented as SEQ ID NO: 3, or an active variant of fragment thereof, wherein the peptide causes an increase in Tau protein in one or more sites in the animal's brain.

Preferably, the method comprises introducing the peptide or variant or fragment thereof into the brain of a wild-type non-human animal. Advantageously, the inventors were surprised to observe that, following administration of the toxic T30 peptide into the brain of a wild-type (i.e. otherwise normal) non-human animal, the total Tau levels were increased in the cortex, subcortex, hippocampus and cerebellum of the animal. Interestingly, the inventors did not observe any significant differences in the levels of 3-amyloid in any region of the dissected brains following the administration of T30. However, previous research (Lin et al, 2009, J. Alzheimer's Dis, 18(4):907-18) has already established that increased total Tau, but not β-amyloid, in CSF correlates with short-term memory impairment in Alzheimer's disease, and, as such, the results described herein are not inconsistent with these earlier findings. Advantageously, therefore, the method of the invention preferably results in the development of a novel animal model of tauopathy, which is indicative of neurodegenerative or neurological disorders.

Accordingly, in a second aspect of the invention, there is provided an animal model for a neurodegenerative disease, which is a non-human animal treated with a peptide comprising or consisting of the amino acid sequence represented as SEQ ID NO: 3, or an active variant of fragment thereof.

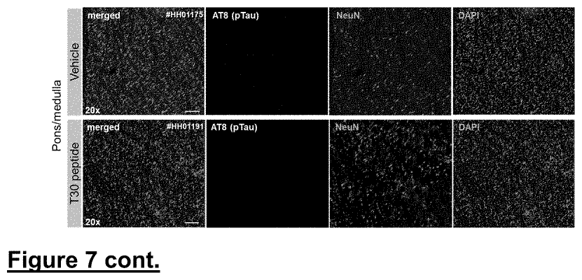

FIG. 3 shows how administration of the T30 peptide surprisingly resulted in: (i) approximately 40-50% increase in Tau in the animal model's cortex, (ii) about 175-200% increase in Tau in the subcortex, (iii) about 30-60% increase in Tau in the hippocampus; and (iv) about 160-180% increase in the cerebellum. The inventors were surprised that such high levels of Tau were achieved upon administration of such low levels of the T30 peptide, i.e. 1 μM or 50 μM. Furthermore, FIG. 8 shows how the administration of the T30 peptide surprisingly decreased the density of NeuN positive cells (i.e. which correlate with mature neurons) in the treated animal's midbrain. As with Tau, the inventors were surprised that such low numbers of NeuN cells or neurones were achieved upon administration of such low levels of the T30 peptide, i.e. 1 μM or 50 PM.

Accordingly, preferably the peptide comprising or consisting of the amino acid sequence represented as SEQ ID NO: 3, or an active variant of fragment thereof, is introduced into the brain of the non-human animal (preferably a normal, wild type animal) in order to create the animal model of the second aspect, which displays an increase in Tau protein in one or more sites in the animal's brain. In addition, preferably the peptide comprising or consisting of the amino acid sequence represented as SEQ ID NO: 3, or an active variant of fragment thereof, is introduced into the brain of the non-human animal (preferably a normal, wild type animal) in order to create the so animal model of the second aspect which displays a decrease in neurons in one or more sites in the animal's brain.

Preferably, administration of the peptide, or variant or fragment thereof to the non-human animal in the method of the first aspect or the model of the second aspect causes an increase in Tau protein or a decrease in neurons in one or more sites in the animal's brain selected from a group consisting of: the cortex; subcortex; hippocampus; cerebellum; basal forebrain; and pons/medulla region. Preferably, administration of the peptide, or variant or fragment thereof causes an increase in Tau protein or decrease in neurons in at least two, three, four, five or all six sites in the animal's brain selected from a group consisting of: the cortex; subcortex; hippocampus; and cerebellum; basal forebrain; and pons/medulla region.

Preferably, administration of the peptide, or variant or fragment thereof causes a statistically significant increase in Tau protein in the one or more sites in the animal's brain compared to an untreated control, preferably at least a 1% increase, or more. Preferably, administration of the peptide, or variant or fragment thereof causes an increase in Tau protein in the one or more sites in the animal's brain by at least 3% compared to an untreated control. Preferably, administration of the peptide, or variant or fragment thereof causes an increase in Tau protein in the one or more sites in the animal's brain by at least 5%, 10% or 20% compared to an untreated control. More preferably, administration of the peptide, or variant or fragment thereof causes an increase in Tau protein in the one or more sites in the animal's brain by at least 30%, 40% or 50% compared to an untreated control.

Preferably, administration of the peptide, or variant or fragment thereof causes a statistically significant decrease in neurons in the one or more sites (and preferably the midbrain thereof) in the animal's brain compared to an untreated control, preferably at least a 1% increase, or more. Preferably, administration of the peptide, or variant or fragment thereof causes a decrease in neurons in the one or more sites (and preferably the midbrain thereof) in the animal's brain by at least 3% compared to an untreated control. Preferably, administration of the peptide, or variant or fragment thereof causes a decrease in neurons in the one or more sites (and preferably the midbrain thereof) in the animal's brain by at least 5%, 10% or 20% compared to an untreated control. More preferably, administration of the peptide, or variant or fragment thereof causes a decrease in neurons in the one or more sites (and preferably the midbrain thereof) in so the animal's brain by at least 30%, 40% or 50% compared to an untreated control.

Acetylcholinesterase is a serine protease that hydrolyses acetylcholine, and will be well-known to the skilled person. The major form of acetylcholinesterase which is found in the brain is known as tailed acetylcholinesterase (T-AChE), and the protein sequence of one embodiment of human tailed acetylcholinesterase (Gen Bank: AAA68151.1) is 614 amino acids in length, and is provided herein as SEQ ID No:1, as follows:

| [SEQ ID No: 1] | |
|---|---|
| 1 | mrppqcllht pslaspllll llwllgggvg aegredaell vtvrggrlrg irlktpggpv |
| 61 | saflgipfae ppmgprrflp pepkqpwsgv vdattfqsvc yqyvdtlypg fegtemwnpn |
| 121 | relsedclyl nvwtpyprpt sptpvlvwiy gggfysgass ldvydgrflv qaertvlvsm |
| 181 | nyrvgafgfl alpgsreapg nvglldqrla lqwvqenvaa fggdptsvtl fgesagaasv |
| 241 | gmhllsppsr glfhravlqs gapngpwatv gmgearrrat qlahlvgcpp ggtggndtel |
| 301 | vaclrtrpaq vlvnhewhvl pqesvfrfsf vpvvdgdfls dtpealinag dfhglqvlvg |
| 361 | vvkdegsyfl vygapgfskd neslisraef lagvrvgvpq vsdlaaeavv lhytdwlhpe |
| 421 | dparlreals dvvgdhnvvc pvaqlagrla aqgarvyayv fehrastlsw plwmgvphgy |
| 481 | eiefifgipl dpsrnytaee kifaqrlmry wanfartgdp neprdpkapq wppytagaqq |
| 541 | yvsldlrple vrrglraqac afwnrflpkl lsatdtldea erqwkaefhr wssymvhwkn |
| 601 | qfdhyskqdr csdl |

The first 31 amino acid residues of SEQ ID No:1 are removed while the protein is released, thereby leaving a 583 amino acid sequence.

The inventor has compared the sequence of β-amyloid (Aβ) with three peptides that are derived from the C-terminus of AChE, which are referred to herein as T30, T14 and T15, and described below.

The amino acid sequence of part of β-amyloid (Aβ) is provided herein as SEQ ID No:2, as follows: —

[SEQ ID No: 2]
DAEFRHDSGYEVHHQKLVFFAEDVGSNKGAIIGLMVGGVVIA

The amino acid sequence of T30 (which corresponds to the last 30 amino acid residues of SEQ ID No:1) is provided herein as SEQ ID No:3, as follows: —

[SEQ ID No: 3]
KAEFHRWSSYMVHWKNQFDHYSKQDRCSDL

The amino acid sequence of T14 (which corresponds to the 14 amino acid residues located towards the end of SEQ ID No:1, and lacks the final 15 amino acids found in T30) is provided herein as SEQ ID No:4, as follows: —

[SEQ ID No: 4]
AEFHRWSSYMVHWK

The amino acid sequence of T15 (which corresponds to the last 15 amino acid residues of SEQ ID No:1) is provided herein as SEQ ID No:5, as follows: —

[SEQ ID No: 5]
NQFDHYSKQDRCSDL

The peptide employed in preparing the animal models of the invention may be derived from acetylcholinesterase itself (i.e. SEQ. ID. No. 1) or an active variant or fragment thereof, including modified forms of that peptide having modified amino acid residues, e.g. a biotinylated form. Variants of the peptide of SEQ ID No:3 include peptides having one, two or three amino acid substitutions and/or one, two or three amino acid deletions and/or one, two or three additional amino acid residues compared to SEQ ID No. 3. A suitable variant may, for example, have an N-terminal and/or C-terminal extension. Given SEQ ID No: 3 as a guide for comparison, it is a straightforward matter to make variant peptides and test them for efficacy in the methods and models according to the invention. For example, one might start by testing a peptide which is identical to SEQ ID No: 3 except for one or two conservatively substituted amino acid residues. Conservative substitutions can be predicted on the basis of amino acid properties which are well characterised. Active variants of SEQ ID No: 3 for use in accordance with the invention may also possibly be determined by in vitro tests of peptides for retention of calcium channel modulatory activity. For this purpose, guinea pig midbrain slices may, for example, be employed for electrophysiological studies as described previously in WO 97/35962. Alternatively, for example, organotypic tissue culture of hippocampal slices, e.g. from rats, may be used.

Preferably, an active variant or fragment of the peptide administered to the brain of the non-human animal comprises or consists of at least 15, 16, 17, 18 or 19 amino acids of the sequence represented as SEQ ID NO: 3. More preferably, an active variant or fragment of the peptide administered to the brain of the non-human animal comprises so or consists of at least 20, 21, 22, 23 or 24 amino acids of the sequence represented as SEQ ID NO: 3. Even more preferably, an active variant or fragment of the peptide administered to the brain of the non-human animal comprises or consists of at least 25, 26, 27, 28 or 29 amino acids of the sequence represented as SEQ ID NO: 3. Preferably, an active variant or fragment of the peptide administered to the brain of the non-human animal comprises or consists of less than 40, 39, 38, 37, 36 or 35 amino acids of the sequence represented as SEQ ID NO: 3. Most preferably, the peptide administered to the brain of the non-human animal comprises or consists of 30 amino acids, i.e. is SEQ ID NO: 3. Suitable variants of SEQ ID No: 3 for use in the invention may be peptides comprising at least 15 amino acid residues and having at least 70% sequence identity with part or all of the AChE sequence of SEQ ID No: 1. Preferably, peptides for use in the invention contain at least 15, 20, 25 or 30 amino acid residues and have at least 90% or 95% sequence identity with SEQ ID No: 3.

The terminals of the peptide or variant or fragment thereof may be protected by N- and/or C-terminal protecting groups with similar properties to acetyl or amide groups. The peptide or variant or fragment thereof may be biotinylated or tritiated. The peptides may be synthetic peptides prepared by chemical synthesis, or they may be prepared from larger peptide or polypeptide molecules by enzymatic digestion, or they may be produced by recombinant techniques.

The method (or assay) comprises administering an effective amount of the peptide comprising or consisting of the amino acid sequence represented as SEQ ID NO: 3, or an active variant or fragment thereof, such that it results in elevated Tau levels in the brain. One or more dosage of the peptide or variant or fragment thereof may be administered to the animal. Preferably, the concentration of the peptide, or variant or fragment thereof, being administered to the animal may be less than 1 mM, or less than 750 μM, or less than 500 μM, or less than 400 μM, or less than 300 μM, or less than 200 μM, or less than 100 μM, or less than 75 μM, or less than 60 μM. Preferably, the concentration of the peptide, or variant or fragment thereof, may be less than 50 PM, or less than 40 μM, or less than 30 μM, or less than 20 μM, or less than 10 PM, or less than 5 μM, or less than 3 M.

Preferably, the concentration of the peptide, or variant or fragment thereof, being administered may be more than 0.01 μM, or more than 0.1 μM, or more than 1 μM, or more than 3 μM, or more than 5 μM, or more than 10 μM. Preferably, the concentration so of the peptide, or variant or fragment thereof, may be more than 20 PM, or more than 30 μM, or more than 40 μM, or more than 50 μM. Preferably, the concentration of the peptide, or variant or fragment thereof, may be more than 60 μM, or more than 70 PM, or more than 80 μM, or more than 90 μM.

It will be appreciated that any of the above concentrations of the peptide, variant or fragment thereof may be combined in any combination. For example, the concentration of the peptide, or variant or fragment thereof, being administered may be between 0.01 μM and 1000 μM, or between 0.1 μM and 500 μM, or between 1 μM and 100 μM, or between 1 μM and 90 μM. Preferably, the concentration of the peptide, or variant or fragment thereof, may be between 0.1 μM and 80 μM, or between 0.1 μM and 70 μM, or between 0.1 μM and 60 μM, or between 0.1 μM and 50 μM. Preferably, the concentration of the peptide, or variant or fragment thereof, may be between 0.1 μM and 40 μM, or between 0.1 μM and 30 μM, or between 0.1 μM and 20 μM, or between 0.1 μM and 10 μM. Preferably, the concentration of the peptide, or variant or fragment thereof, may be between 10 μM and 80 μM, or between 20 μM and 80 μM, or between 30 μM and 70 μM, or between 40 μM and 60 μM. In a most preferred embodiment, about 1 μM or 50 μM of T30 or variant or fragment thereof is administered to the brain of the non-human animal. Accordingly, any of the above upper and lower limits may be combined with each other.

FIG. 8 shows how the administration of the T30 peptide (50 PM) surprisingly decreased the density of NeuN expressing cells (i.e. which correlate with mature neurons) in the treated animal's midbrain. As shown in FIGS. 2 and 3, administration of the T30 peptide induces a highly significant, dose-dependent increase in Tau in all four brain areas studied. The highest dose tested (i.e. 100 μM) showed no difference in Tau concentration compared to PBS-injected controls. Although not wishing to be bound any hypothesis, the inventors believe that this dose-dependent effect may be due to a shutting down of the calcium channel when excessively stimulated. However, in lower doses (i.e. less than 100 μM), where the enhanced calcium influx is viable, the T30 peptide induces activation of glycogen synthase kinase 3 (GSK3) which results in an increased phosphorylation of Tau, which in turn promotes the formation of Tau tangles in the brain, which is the cardinal marker of AD. In other words, the inventors have surprisingly shown that lower μM doses of T30 (i.e. less than 100 μM) are clearly receptor-mediated, whereas high doses (i.e. above 100 μM) are not receptor-mediated, which was totally unexpected. The inventors believe, therefore, that the dose range of 0.1-99 μM T30 peptide or fragment or variant thereof, at which it is receptor-mediated is optimum, and therefore preferred.

The peptide, or variant or fragment thereof may be introduced into the basal forebrain region of the brain. The peptide, or variant or fragment thereof may be introduced into the medial septum/diagonal band of Broca (SID13) region of the brain. The peptide, or variant or fragment thereof may be introduced into the cortical cholinergic system.

Both the cortical and septohippocampal cholinergic systems contribute to memory, and are therefore preferred sites for administering the peptide. However, preferably the peptide, or variant or fragment thereof may be introduced into the nucleus basalis magnocellularis (NBM).

The peptide may be administered by stereotaxic injection into an anaesthetised animal, although administration to conscious animals through implanted cannulae may sometimes be preferred, e.g. to examine acute effects (30 minutes duration) without anaesthesia. Alternatively, pressure microinjection or electrophoresis through a (e.g. glass) micropipette may be preferable for ionophoresis recordings.

Preferably, the non-human animal is a normal, wild type non-human animal. For example, the animal may be a mammal, which may be a primate, for example, a monkey. The non-human animal may be male or female. Preferably, however, the non-human animal is a rodent, which may be a mouse or a rat. Preferably, the rodent is a rat. The rat may be a Lister hooded rat or a Long Evans hooded rat. The rat may be male or female, but is preferably male. The rat may be an adult rat, i.e. at least 2 or 3 months old. Preferably, the non-human animal is a normal, wild type rodent.

Preferably, the peptide, or a variant or fragment thereof contributes to, or causes, neurodegeneration. The peptide or variant or fragment thereof administered to the animal model preferably causes cellular degeneration and thereby impairment of a testable brain function, wherein impairment of the same brain function in a human is indicative of a neurological disorder.

For example, the models or methods described herein may be used to investigate any neurodegenerative disease which is characterised by tauopathy. For example, the neurodegenerative disease may be selected from a group consisting of: Alzheimer's disease; Parkinson's disease; Motor Neurone disease; Spinocerebellar type 1, type 2, and type 3; Amyotrophic Lateral Sclerosis (ALS); Lewy-body dementia; and Frontotemporal Dementia. It is preferred that the model is used to study any neurological disorder associated with non-enzymatic function of acetylcholinesterase, in particular, Alzheimer's Disease, Parkinson's Disease and Motor Neuron Disease.

However, it is especially preferred that the model or method is used to study Alzheimer's Disease. Accordingly, the testable brain function, the impairment of which may be tested, may be cognitive function. Alternatively, or additionally, the impairment may be an attentional deficit. Preferably, the method comprises testing the animal model for the impairment of an appropriate brain function, for example by providing the animal with an attentional task to test the attentional impairment.

Peptide-treated animal may be tested for one or more impairment of memory, learning, attention and/or problem solving. Preferred methods for testing animals for cognitive function are working spatial memory tests, such as the T maze test (Rawlins et al., 1982, Beh. Brain Res, 5, 331-358). Other standard tests which may be used include the Morris water maze (Morris et al., 1982, Nature, 297, 681-683) and the radial arm maze (Olton et al, 1976, Animal Beh. Proc. 2, 97-116).

Preferably, the method comprises combining the production of peptide lesions in the brain (e.g. basal forebrain) with testing for attentional deficit using apparatus which provides a serial choice reaction task. Rats can be trained to perform simple attentional tasks, such as to push open a panel with their nose when a light flashed behind it to retrieve a food reward. Although sensitive to treatments affecting attention, a failure to respond in such a test might also be due to an effect on performance. The treatment might for example cause sedation. A serial choice reaction task addresses this concern by providing more than one stimulating event, for example lever-pressing by a rat may result in one of three events: a light flash from a left magazine or a right magazine or no light in which case the correct choice is a central magazine. Suitable apparatus for testing for attentional impairment in this manner has been described in Higgs et al., European J. Neuroscience (2000) 12, 1781-1788.

Other behavioural functions which may be monitored include but are not limited to social behaviour, emotional reactivity, contextual conditioning, pre-pulse inhibition of startle reflex, two-way aversive conditioning and motivation as measured by food and water intake or sucrose preference.

As indicated above, subtle lesions in the brain of rats giving rise to attentional deficit have been found to be achievable by use of the peptide of SEQ ID No:3. However, it is envisaged that functionally equivalent lesions in the NBM may be achieved by use of other peptides as discussed above.

The animal models and methods described herein can be used to examine many molecular processes relating to tauopathy and associated neurodegenerative disorders, test pharmacological compounds which may regulate these processes and provide a reliable tool for drug screening.

Hence, preferably the method further comprises administering prior to, simultaneously or after the peptide, or variant or fragment thereof, a test agent and determining whether the agent can inhibit, prevent or increase impairment of the testable brain function and/or can inhibit, prevent or increase cellular damage in the brain. Preferably, the test agent is selected, which is a compound capable of inhibiting or preventing impairment of the testable brain function. Preferably, the method further comprises synthesising the test compound.

Thus, in a third aspect of the invention, there is provided a use of the non-human animal model according to the second aspect, or prepared in accordance with the method of the first aspect, to: (i) examine neurodegenerative or neuroregenerative processes; (ii) test pharmacological compounds which may modulate neurodegenerative or neuroregenerative processes; or (iii) screen neurodegenerating or neuroregenerating drugs.

Modulation of neurodegeneration may include inhibition, prevention or increasing neurodegeneration.

In a fourth aspect, there is provided a method of identifying a candidate agent, for use in the treatment, prevention or amelioration of neurodegenerative disorder, the method comprising:
administering a candidate agent to the animal model according to the second aspect, or prepared in accordance with the method of the first aspect; and
determining if the candidate agent inhibits, prevents or increases impairment of so a testable brain function and/or causes improvement or deterioration of cellular damage in the brain,

wherein inhibition or prevention of impairment of the testable brain function, or improvement of cellular damage in the brain indicates that the test agent is a candidate for the treatment, prevention or amelioration of neurodegenerative disorder, whereas increasing impairment of the testable brain function or deteriorating cellular damage in the brain indicates that the agent is not a candidate for the treatment, prevention or amelioration of neurodegenerative disorder.

Cellular damage may comprise neurodegeneration. Such damage may be monitored or assessed by measuring one or more of:
(i) the inhibition of activity in neuronal populations (i.e. assemblies);
(ii) calcium levels;
(iii) acetylcholinesterase activity levels;
(iv) expression of alpha-7 nicotinic receptors in cell membranes; and
   (v) cell density and/or loss or reduction of NeuN-expressing cells (related to neuronal death) in specific areas.

Preferably, the testable brain function may be a cognitive function or an attentional deficit. Preferably, the method comprises testing the animal model for impairment or a cognitive function or an attentional deficit.

In a fifth aspect, there is provided a method of testing a test agent for biological activity in a neurodegenerative disease, wherein the method comprises administering the test agent to an animal model according to the second aspect or prepared by the method of the first aspect, and assessing the animal having a brain lesion for any change, either improvement or deterioration, associated with the brain lesion.

Such assessment will comprise determining whether said agent will inhibit, prevent or increase impairment of an appropriate testable brain function, e.g. a cognitive function such as attention or memory, and/or determining whether there is any improvement or deterioration in cellular damage at the relevant site(s) in the brain. The test agent is preferably a drug compound.

The following is a list of some other behavioural tests which will be suitable for use in so accordance with the invention. Most but not all of these are tests of cognitive function.

Tests which relate to behaviour but not cognitive faculties are also included and may be used instead of or in addition to the tests of cognitive function such as memory.

Attention
Carli, M., Robbins, T. W., Evenden, J. L. and Everitt, B. J. (1983) Effects of lesions to ascending noradrenergic neurones on performance of a 5-choice serial reaction time task in rats-, implications for theories of dorsal noradrenergic bundle function based on selective attention and arousal (Behavioural Brain Research 9, 361-380).

Social Behaviour
Gardner, C. R. and Guy, A. P. (1984) A social interaction model of anxiety sensitive to acutely administered benzodiazepines. Drug Dev. Res. 4, 207216.

Emotional Reactivity
Gray, J. A. (1982) The neuropsychology of anxiety Dawson, G. R. and Tricklebank M. D. (1995) Use of the elevated plus maze in the search for novel anxiolytic agents. TIPS 16, 33-36.

Morris Water Maze
Morris, R. G. M., Garrud, P., Rawlins, J. N. P. and O'Keefe, J. (1982) Place navigation impaired in rats with hippocampal lesions. Nature 297, 681-683.

Radial Arm Maze
Olton, D. S. and Samuelson, R. J. (1976) Remembrance of places past: 20 spatial memory in rats. Journal of Experimental Psychology: Animal Behaviour Processes 2, 97-116.

T Maze
Rawlins, J. N. P. and Oiton, D. S. (1982) The septo-hippocampal system and cognitive mapping. Behavioural Brain Research 5, 331-358.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified herein.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein, i.e. SEQ ID No:1-5.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: —(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences so is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula: —Sequence Identity=(N/T)*100.

Alternative methods for identifying similar sequences will be known to those skilled in the art. For example, a substantially similar nucleotide sequence will be encoded by a sequence, which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in SEQ ID No: 1-5.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing so a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which: —

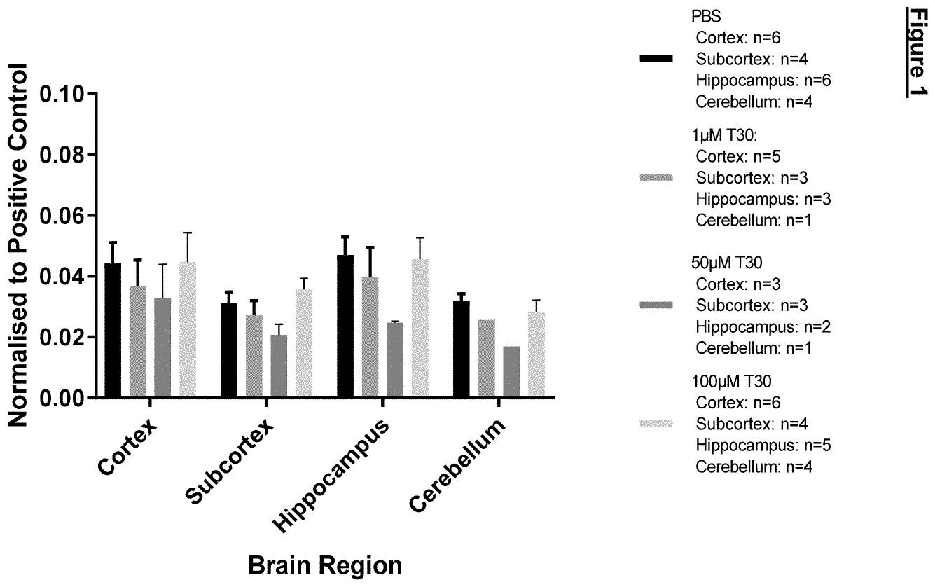

FIG. 1 shows β-Amyloid (42) levels in dissected rat brain (cortex, subcortex, hippocampus and cerebellum) following injection of PBS (control) or 1 μM, 50 μM or 100 μM T30 (treatments) into the basal forebrain;

FIG. 2 shows total Tau levels in dissected rat brain (cortex, subcortex, hippocampus and cerebellum) following injection of PBS (control) or 1 μM, 50 μM or 100 μM T30 (treatments) into the basal forebrain;

FIG. 3 shows the percentage of Tau in dissected rat brain areas (cortex, subcortex, hippocampus and cerebellum) after injection of PBS (control) or 1 μM, 50 μM or 100 μM T30 (treatments) into the basal forebrain;

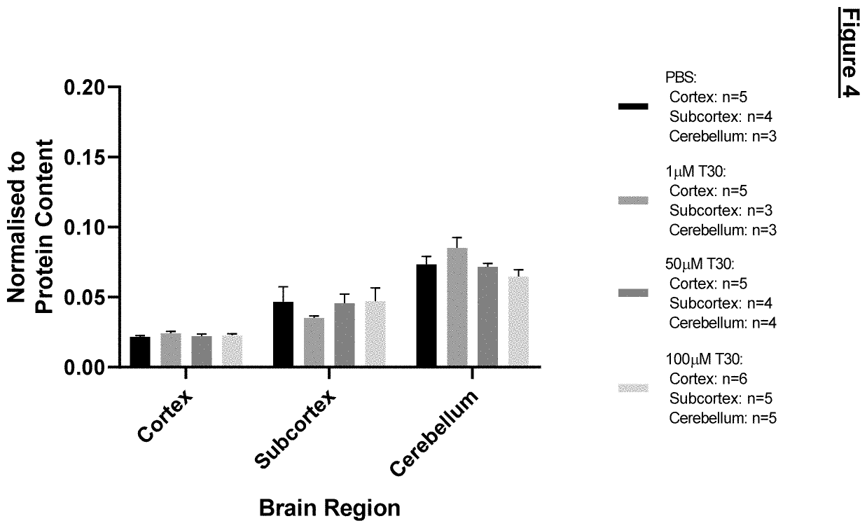

FIG. 4 shows T14 levels in dissected rat brain (cortex, subcortex and cerebellum) following injection of PBS (control) or 1 μM, 50 μM or 100 μM T30 (treatments) into the basal forebrain;

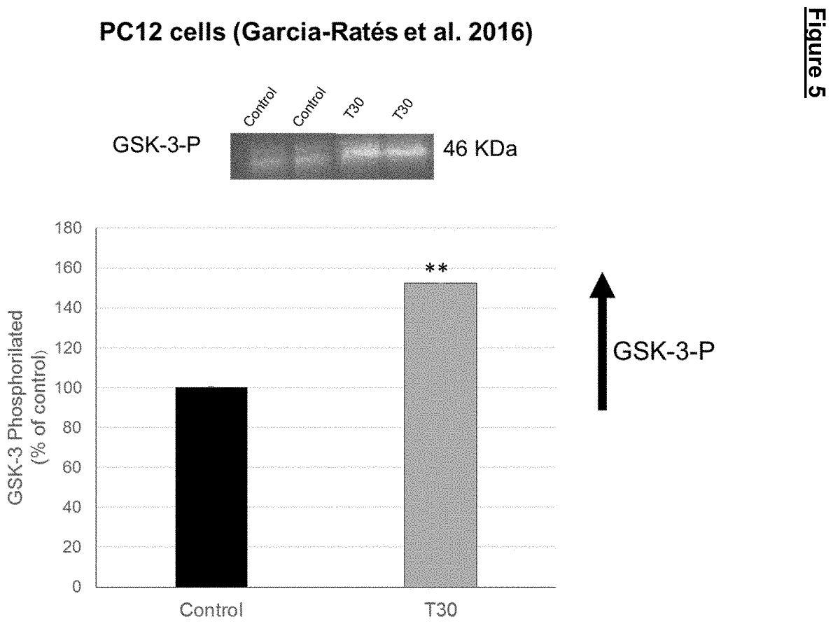

FIG. 5 shows data taken from a paper (Garcia-Rates et al., 2016, “(I) Pharmacological profiling of a novel modulator of the α7 nicotinic receptor: Blockade of a toxic acetylcholinesterase-derived peptide increased in Alzheimer brains”, Neuropharmacology, 2016, June, 105: 487-499) which shows that lower doses of T30 results in calcium influx in PC12 cells, which in turn causes glycogen synthase kinase 3 (GSK3) levels to;

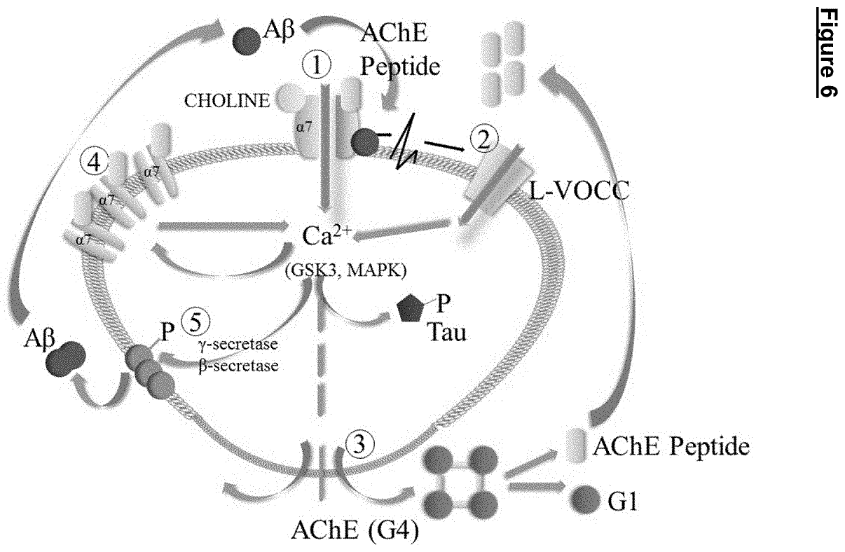

FIG. 6 shows the cascade of events resulting from the effect of T30 in a cell;

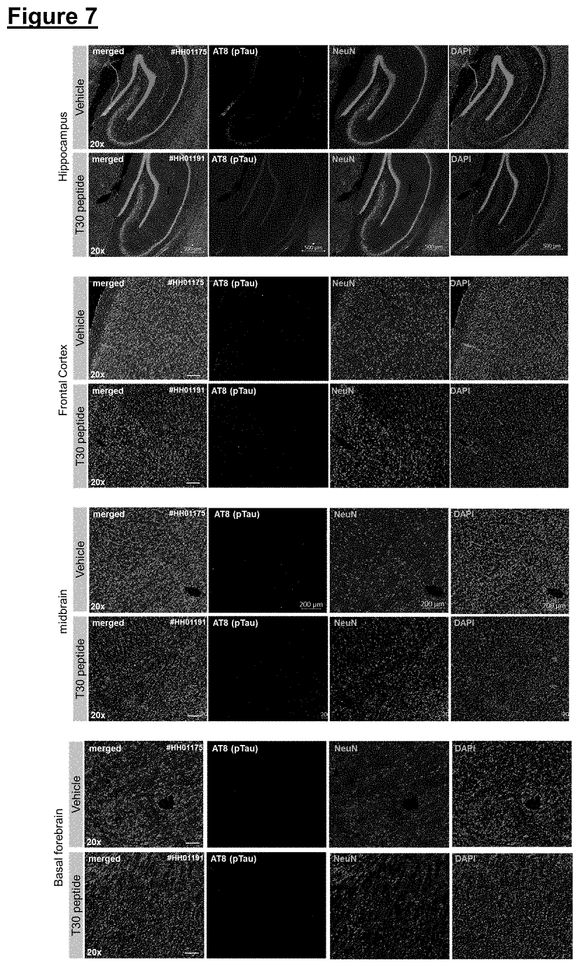

FIG. 7 shows IHC effects of AChE-Derived Peptide (T30) on Alzheimer's Disease Related Parameters (pTau, NeuN) in Sprague-Dawley Rats. Immunohistochemical staining of sections from WT Sprague Dawley rats following acute treatment with T30 peptide or saline. No intracellular pTau (yellow) is detected in hippocampus, cortex, midbrain, basal forebrain or pons/medulla. NeuN (green) is used to detect neurons and nuclei are detected with DAPI (blue);

FIG. 8 shows quantitative analysis of Effects of AChE-Derived Peptide (T30) on Alzheimer's Disease Related Parameters (pTau, NeuN) in Sprague-Dawley Rats. Quantification of the density of NeuN positive cells in cortex, hippocampus, midbrain, pons/medulla and basal forebrain of WT Sprague Dawley rats after treatment with T30 peptide or saline. Statistical analysis was performed using an unpaired t test. *p<0.05; **p<0.01 vs. saline; n=8, T30 peptide, n=8; 6 sections per animal;

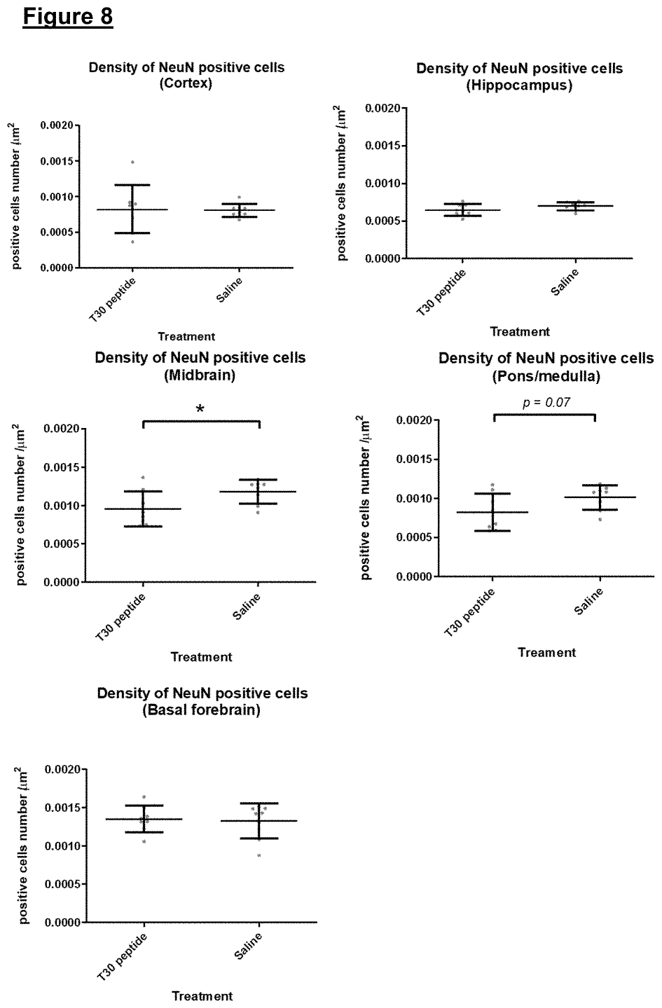

FIG. 9 shows IHC effects of AChE-Derived Peptide (T30) on Alzheimer's Disease Related Parameters (6E10, Iba1) in Sprague-Dawley Rats. Immunohistochemical staining of sections from WT Sprague-Dawley rats following treatment with T30 peptide or saline. No intracellular Aβ deposits (yellow) is detected in hippocampus, cortex, midbrain, basal forebrain, and pons/medulla. Microglia are detected with Iba1 (green) and nuclei are detected with DAPI (blue);

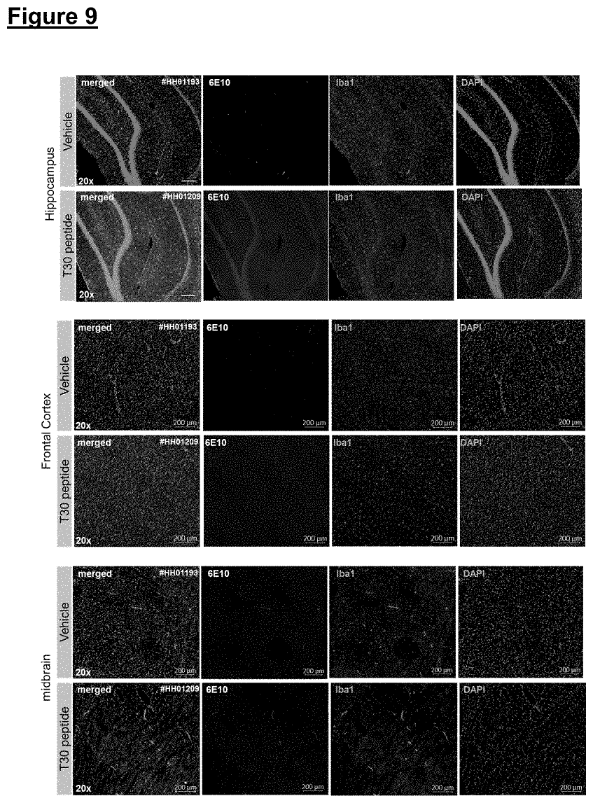

FIG. 10 shows quantitative analysis of Effects of AChE-Derived Peptide (T30) on Alzheimer's Disease Related Parameters (pTau, NeuN) in Sprague-Dawley Rats. Quantification of the density of Iba1 positive cells in cortex, hippocampus, midbrain, pons/medulla and basal forebrain of WT Sprague Dawley rats after treatment with T30 peptide or saline. Statistical analysis was performed using an unpaired t test. *p<0.05; **p<0.01 vs. saline; n=8, T30 peptide, n=8; 6 sections per animal;

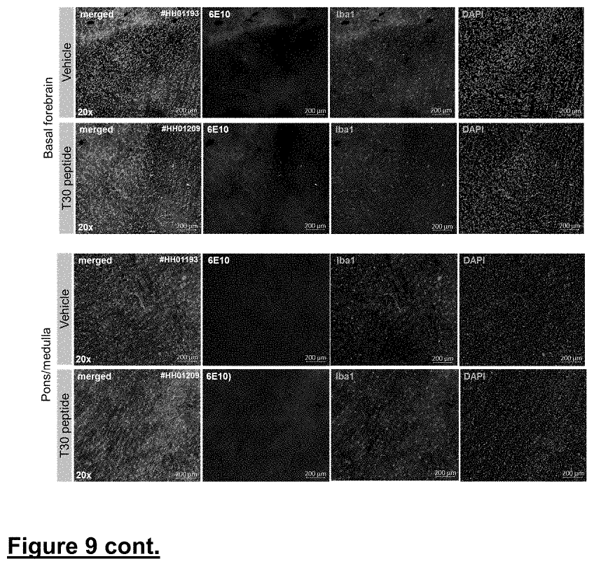

FIG. 11 shows the results of the Morris Water Maze (MWM) Quadrant & Platform Timepoint 1 experiment;

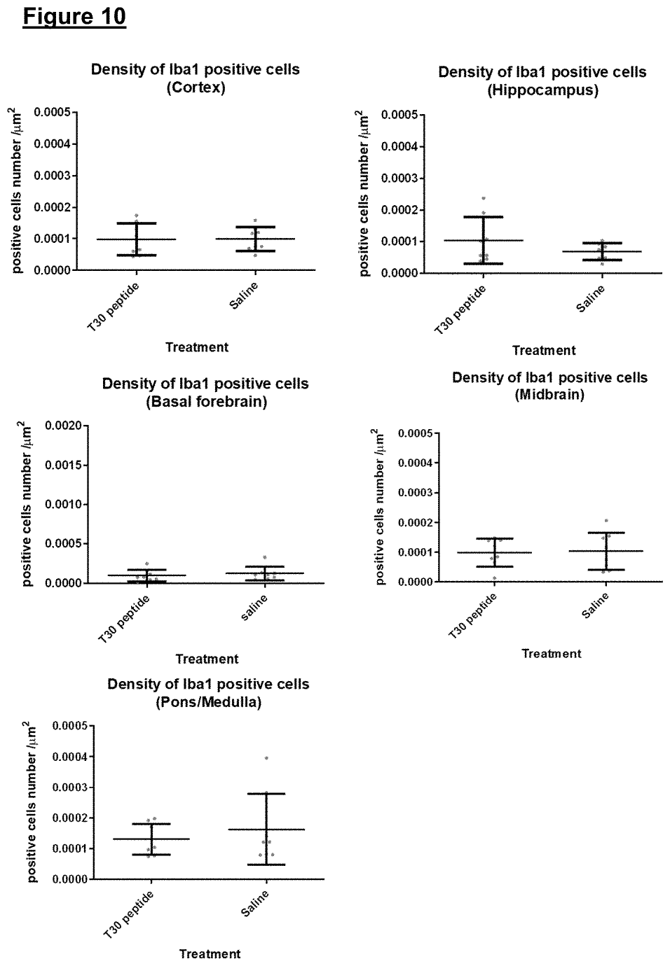

FIG. 12 shows the results of the Morris Water Maze (MWM) Quadrant & Platform Timepoint 2 experiment;

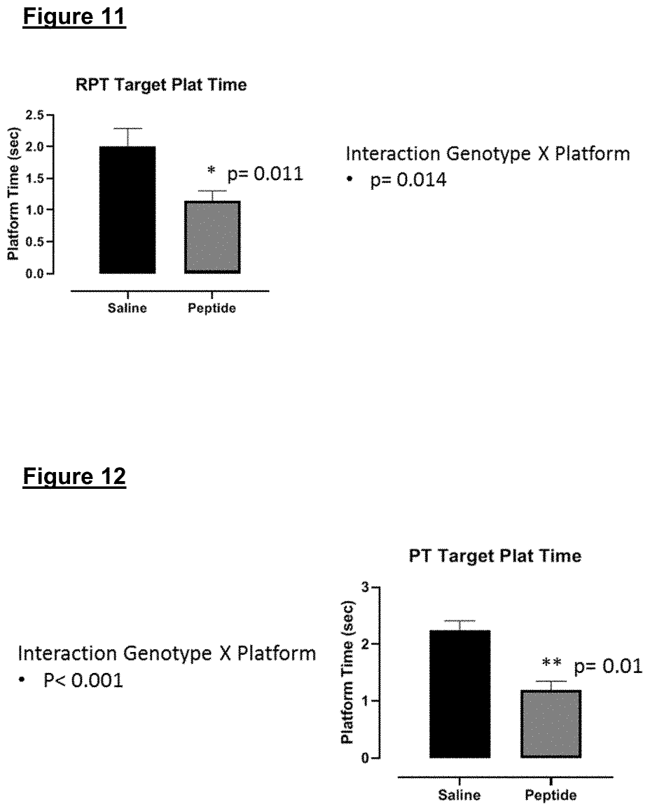

FIG. 13 shows the results of the Morris Water Maze (MWM) Quadrant & Platform Timepoint 1, 2 and 3 experiment;

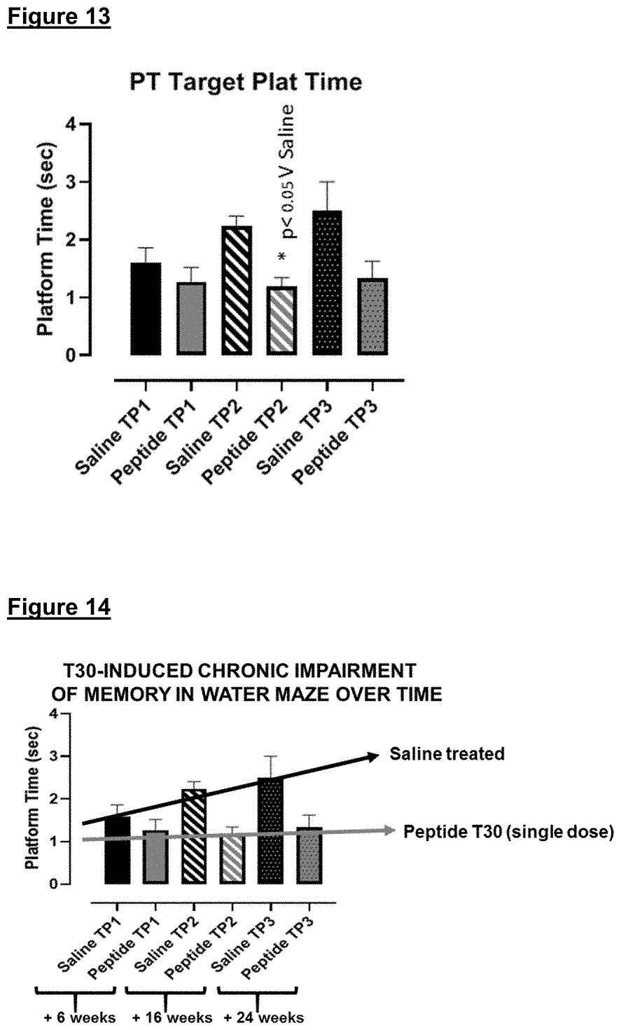

FIG. 14 shows T30-induced chronic impairment of memory in water maze over time; and

FIG. 15 shows the effects on normal rats of a single intracerebral injection of T30 at 3 weeks, 16 weeks and 24 weeks post administration.

### EXAMPLES

Despite the increasing numbers of studies targeting the primary events in neurodegeneration, there is no animal model which closely reproduces the full pathological profile (e.g. of Alzheimer's disease), since the basic mechanisms of neurodegeneration are still poorly understood. Thus, the inventors have developed a novel in vivo animal model to elucidate the basic mechanisms inducing neurodegeneration, and, importantly, in which novel test agents could be tested to determine neuroprotective (or neurotoxic) activity.

The invention involves the use of a peptide cleaved from the C-terminus of acetylcholinesterase (AChE), T30 (SEQ ID NO:3), which is composed by a bioactive portion, T14 (SEQ ID No: 4), and an inert fragment, T15 (SEQ ID No: 5) that interacts with the α7 nicotinic acetylcholine receptor (α7-nAChR). The inventors have previously shown that the application of AChE-derived peptide on cell lines promotes an AD-like phenotype. These effects are blocked by a novel candidate modulator of the α7-nAChR, NBP-14, which is the cyclized form of T14, and so has the sequence of cyclic SEQ ID No:4. As described below, the inventors have applied T30 or NBP14 on ex vivo brain slices and investigated their activity in modulating the endogenous T14 expression, and whether they contribute or prevent a neurodegeneration pattern.

The inventors show that the apparatus and model can be used to study neurodegeneration in a more physiological scenario, i.e. ex vivo brain slices, on α7-nAChR, p-Tau and Aβ expression, though it will be appreciated that there any many other proteins that can be measured to monitor degree and progression of neurodegenerative disorders. The model harnesses the inventors' new hypothesis which they believe accounts for the aberrant processes characterizing AD, based on the interaction between the α7 nicotinic acetylcholine receptor (α7-nAChR) and the toxic peptide, cleaved from the acetylcholinesterase (AChE) C-terminus, i.e. T30. The apparatus and models can be used to examine many molecular processes, test pharmacological compounds which may regulate these processes and provide a reliable tool for drug screening, reducing whole animal experiments.

Materials and Methods

Brain Extraction and Dissection

Following a lethal injection of anaesthesia (pentobarbital), fresh brains were extracted and the cortex, hippocampus, cerebellum and subcortical areas were dissected and immediately snap frozen in liquid nitrogen. Brains were stored at −80° C. to preserve the proteins. Due to death of one rat prior to the experiment beginning, the groups were as follows:
PBS (control): n=6
1 μM T30: n=5
50 μM T30: n=6
100 μM T30: n=6

Brain Homogenisation

Brain sections were defrosted on ice, and ice cold Lysis Buffer (PBS+protease and phosphatase inhibitors at 1:100 each) was added to each brain section. Using a pestle, the tissue was homogenised as much as possible before a sonicator probe was used on a low setting, for 5 seconds at a time, whilst being kept on ice, until the tissue was fully homogenised. Samples were incubated on ice for 20 minutes before being centrifuged at maximum speed (13,000 rpm) for 30 minutes at 4° C. The supernatant was removed and used for analysis.

β-Amyloid ELISA Commercial ELISAs for β-Amyloid 42 (Invitrogen, KMB3441) were purchased along with β-Amyloid Peptide (1-42) (Abcam, ab120959). All samples were plated at 6000 μg/mL of total protein (determined by the Pierce Protein Assay) with a positive control synthesized from wild type, whole rat brain plus β-Amyloid Peptide (1-42) at 275 ng (published concentrations found in Transgenic Animal Models of AD).

Secondary controls (no primary antibody added) and chromogen blanks were also used on every plate. A standard curve of β-Amyloid Peptide (1-42) was used ranging from 0-200 pg/mL on every plate and the protocol was followed as set out by the kit (with the exception of the peptide supplied with the kit, which was replaced by an alternative, listed above). Roughly, standards (in duplicate) and samples (in triplicate) were plated and incubated at room temperature on a plate shaker for 2 hours. All standards and samples were aspirated and the plate washed before the ‘detector’ antibody supplied with the kit was added to all wells with the exception of secondary controls and chromogen blanks. A further 1 hour incubation period at room temperature on a plate shaker followed, before the antibody was aspirated and the plate washed again. IgG HRP was added to every well (with the exception of the chromogen blanks) and the plate incubated for a further 30 minutes at room temperature on a plate shaker. All solution was aspirated and the plate washed before adding Stabilized Chromogen to every well and incubating the plate for 30 minutes in the dark, at room temperature on a plate shaker. Finally, Stop Solution was added to every well and the absorbance read at 450 nm.

Total Tau ELISA

Commercial ELISAs for Total Tau detection (Abcam, ab210972) were purchased. All samples were plated at 0.5 μg/mL of total protein (determined by the Pierce Protein Assay) along with a full standard curve of Tau ranging from 0-2000 pg/mL and Secondary controls (no primary antibody added). The protocol was followed as set out by the kit, roughly, standards (in duplicate) and samples (in triplicate) were plated, followed immediately by the addition of the Antibody Cocktail (minus the Capture Antibody for the Secondary Controls) and incubated at room temperature on a plate shaker for 1 hour. All solution was aspirated and the plate washed before TMB Substrate was added to all wells and incubated for 10 minutes in the dark, at room temperature on a plate shaker. Finally, Stop Solution was added to all wells and the plate incubated for 1 minute at room temperature on a plate shaker before the absorbance was read at 450 nm.

T14 ELISA

The inventors have developed an in-house ELISA for the detection of T14. All samples left (PBS: cortex n=6, subcortex n=6, hippocampus n=0, cerebellum n=4; 1 μM T30: cortex n=5, subcortex n=3, hippocampus n=1, cerebellum n=4; 50 μM T30: cortex n=5, subcortex n=4, hippocampus n=1, cerebellum n=4; 100 μM T30: cortex n=6, subcortex n=6, hippocampus n=0, cerebellum n=6) were diluted to 1:10 and plated (in triplicate) with a full T14 standard curve (plated in duplicate) ranging from 0-40 nM and Secondary Controls. Plates were incubated overnight at 4° C. on a plate shaker and then fully aspirated before addition of BSA Blocking Solution and a further incubation of 6 hours at room temperature on a plate shaker. Blocking Solution was aspirated and primary antibody (T14 Polyclonal, Genosphere) added to all wells (with the exception of Secondary Controls) before incubating overnight at 4° C. on a plate shaker. Antibody solution was aspirated and the plate washed followed by the addition of secondary antibody and incubation for 2 hours at room temperature on a plate shaker. All solution was aspirated and the plate washed, then TMB substrate was added and the plate incubated for 15 minutes at room temperature on a plate shaker. Stopping Solution was added and the absorbance read at 450 nm.

Tissue Preparation and Immunohistochemistry

Rat brain samples were removed from PBS and cryoprotected by incubating in 30% sucrose solutions for 72 h or until saturated. Whole brains were cut in half along the midline and each half was embedded in TissueTek and stored at −80° C. until the time of cyro-sectioning.

Sagittal sections of 25 μm were cut using a cryostat starting at the midline. Sections were collected in 24-well plates, and directly used for staining or stored in a cryoprotection solution (25 mM Na-phosphate buffer pH 7.4, 30% ethylene glycol, 20% glycerol) at −20° C. until time of use. All staining were performed with sections mounted on superfrost slides.

Immunostaining for the detection of beta amyloid (Aβ), phosphorylated Tau (pTau), neurons (NeuN) and microglia (Iba1) was performed in the following manner. Sections were pretreated for antigen retrieval either in citric Buffer pH 6.0 for 30 minutes at 90° C. for pTau or with 70% formic acid for 10 min for Aβ. After antigen retrieval sections were permeabilized in 0.3% Triton X-100/PBS, blocked in 10% normal goat serum/PBS and incubated with the primary antibody diluted in 1% normal goat serum, 0.1% Triton X-100 in PBS at 4° C. overnight.

The following primary antibodies were used for immunostaining: anti-beta amyloid (Aβ) monoclonal mouse, 6E10, (1:1000; Covance, cat #39320), monoclonal mouse anti-phosphorylated Tau, AT180, (1:500; Thermo, cat #MN1040), monoclonal rabbit anti-Iba1 (1:500; Synaptic System, cat #234004), polyclonal rabbit anti-NeuN (1:500; Millipore, cat #ABN78).

Co-stainings were performed with 6E10 combined with Iba1 and AT180 combined with NeuN. Sections were washed three times in PBS for 15 min and incubated in appropriate secondary antibody (Sigma) for 2 hours at room temperature. Sections were again washed in PBS three times in PBS for 15 min, then incubated with DAPI staining to detect nuclei. Finally, mounting media was applied to stained sections and slides were coverslipped for imaging with the Zeiss AxioScan.Z1 system (Carl Zeiss Microscopy).

Image Acquisition and Quantitative Analysis

Automated image acquisition was conducted using a Zeiss AxioScan.Z1 slide scanning device (Leica Biosystems) equipped with an LED-Colibri7 light source and an Axiocam 506 mono camera set. Images were taken with 20× magnification (pixel size: 0.22 μm) in a none-confocal manner and images were visualized using Zen software. Image data was imported into the Visiopharm® image analysis software (Visiopharm A/S) to perform region selection.

Manual segmentation of the cortex, hippocampus, midbrain, basal forebrain and pons/medulla regions was performed by subdividing the images of the sagittal brain sections using coordinates published by the Allen Developing Mouse Brain Atlas (Allen Institute) as guidelines.

Image analysis scripts for characterization and quantification of intracellular and extracellular Aβ, pTau, NeuN and Iba1 were developed using Acapella® Studio 5.1 (PerkinElmer Inc.) and the integrated Acapella® batch analysis as part of the Columbus® system. For all analyses individual cells within tissue sections were identified using the DAPI signal and a customised nuclei detection workflow based on the Acapella® “nuclei_detection_B” algorithm. Several quality control parameters were implemented to discard out out-of-focus nuclei and non-nuclear structures. These include e.g. applying thresholds for minimum signal contrast, nuclear area and nuclear roundness. Cytoplasm of detected cells was defined as a 4-pixel-wide concentric ring around the previously segmented nuclei (perinuclear area). Outside this perinuclear ring, a 3-pixel-wide background area was created, serving as cellular individual and, after median aggregation, whole-brain-region reference region for determination of NeuN- and Iba1-positive cellular populations.

Signal intensities for Aβ, pTau, NeuN and Iba1 stainings were evaluated in all cellular sub regions. Cells were identified as being NeuN- or Iba1-positive when the average signal intensity in the nuclear area was at least 1.5 or 2 times higher than the brain region median background, respectively.

Extracellular plaques were segmented by applying an intensity threshold to the image: signal with at least 2 times higher intensity than the median cellular amyloid background was considered potentially belonging to a plaque. To exclude false-positive plaques from analysis, further filtering of these initial plaque-like objects was achieved by applying thresholds for minimum plaques size (i.e. >200 p×2) and axial ratio (length small axis/length long axis >0.4). All readouts were calculated as average values per brain region and histological section. These values were then used to calculate respective averages per animal.

Data Handling and Analysis

A total of 16 animals were used for the study, with N=8 animals per treatment group.

Quantitative results for six sections per animal were averaged to generate one data point per animal. Statistical analysis was performed using an unpaired t test. *p<0.05; **p<0.01 T30 peptide vs. saline.

Antibodies Used for Immunohistological Analysis of Brain Samples of Sprague-Dawley Rats.

| AD related pathology | Phenotype detected | Primary antibody |
|---|---|---|
| Aβ Plaque | Beta amyloid | 6E10 |
| Tau | Phosphorylated tau | AT8 |
| Gliosis | Activated microglia | Iba1 |
| Cell loss | Neronal cell count | NeuN |

Analysis

Initially, the standard deviation of the blanks, Limit of Detection (LOD) (standard deviation of the Blanks×3.3) and Limit of Quantification (LOQ) (standard deviation of the Blanks×10) were calculated from the absorbance values (A₄₅₀). If applicable, the average of the Chromogen Blanks was taken away from all standard curve, sample and control values, followed by the average of the Blanks and then the average of the Secondary Controls. All values above the LOQ were used to plot graphs and interpolate values (if applicable) as pg/mL using GraphPad Prism Software. All statistical analysis was performed using GraphPad Prism Software.

Human Tau SimpleStep ELISA Kit—Abeam ab210972:

The protocol is as follows:
Prepare all reagents, working standard, and samples.
Remove excess microplate strips from the plate frame, return them to the foil pouch containing the desiccant pack, reseal and return to 4° C. storage.
Add 50 μl of all sample or standard to appropriate wells.
Add 50 μl of the antibody cocktail to each well.
Seal the plate and incubate for 1 hour at room temperature on a plate shakers set to 400 rpm.
Wash each well with 3×350 μl 1× wash buffer PT. Wash by aspirating or decanting from wells and then dispensing 350 μl 1× wash buffer PT into each well. Complete removal of liquid at each step is important for good performance. After the last wash, invert the plate and blot it against clean paper towels to remove excess liquid.
Add 100 μl of TMB substrate to each well and incubate for 10 minutes in the dark on a plate shaker set to 400 rpm.
Add 100 μl of stop solution to each well. Shake plate on a plate shaker for 1 minute to mix. Record the OD at 450 nm, and this is an

In addition, secondary controls were added to all plates which were subtracted from all A₄₅₀ Values during the normalisation.
Peptide for the standard Curve, dilution of the standards and all samples were diluted in 1× Cell Extraction Buffer (5× Cell Extraction Buffer PTR provided with the kit) plus 1× Cell Extraction Enhancer Solution (50× Cell Extraction Enhancer Solution provided with the kit) in dH₂O.
1× Wash Buffer prepared by diluting 10× Wash Buffer PT (provided with the kit) with dH₂O.
Antibody Cocktail:
   1× Human Tau Capture Antibody+1× Human Tau Detector Antibody (both provided with kit in 10× form) diluted in Antibody Diluent CPI (provided with the kit).
Antibody for Secondary Controls:
   1× Human Tau Detector Antibody (provided with kit in 10× form) diluted in Antibody Diluent CPI (provided with the kit).

Statistical Analysis:
Average A₄₅₀ of the Blanks was subtracted from all Standard and Sample A₄₅₀ Values
Average of A₄₅₀ of the Secondary Controls was subtracted from all Standard and Sample A₄₅₀ Values
An Ordinary One Way ANOVA was performed on each brain area against the Control for that area, with Dunnett's Multiple Post-Hoc Comparisons test.

Morris Water Maze Method

The 2.1 m diameter black water maze pool is filled to a depth of 40 cm with 22 degree C. water. This leaves the 15-cm diameter submerged platform 1 cm below the water level. The rat is then placed in the water at one of the cardinal points (N, E, S, W) quadrant and allowed 2 minutes to find the platform. If the rat finds the platform within this time it is allowed 15 seconds on the platform before it is removed, gently towelled down and placed under a warming lamp. If the rat does not find the platform within the 2 minutes it is led to the platform by trailing a hand in the water in front of the rat, leading it to the platform. It is then allowed 15 seconds on the platform before it is removed, towelled down and placed under a warming lamp. The routine is repeated 4 times per day (maximum 10 days, although the current quote allows for 6 days of testing with 4 days of reversal learning) until the rat has clearly learnt the maze, signified by no significant improvement occurring after 3 consecutive days. The inter-trial interval time between swims is 10 minutes, A probe trial is run at the end of both reference memory trials and reversal learning trials to probe working memory.

### Example 1

The primary objective was to establish whether a single dose of T30, injected into the basal forebrain of WT rats, could induce, neurochemically, an ‘Alzheimer's-like’ profile, defined as statistically significant increases in AD-related proteins in treatment groups compared to control. Secondly, the objective of this work was to establish at which concentration T30 caused these changes.

Stereotaxic injection of either PBS (control), or one of 3 doses of T30 (1 M, 50 μM and 100 μM) into MS/VDB (Medial Septum/Vertical Limb of the Diagonal Band) of adult male Lister hooded rats was performed at Nottingham University. Rats were culled 2-3 weeks after injection and brains were extracted and dissected to separate cortex, hippocampus, cerebellum and subcortical areas for neurochemical analysis at Neuro-Bio. Each brain area was analysed for levels of total Tau, β-Amyloid 42 and T14.

Results

### Example 1—β-Amyloid (42)

Referring to FIG. 1, due to the difficulty detecting β-Amyloid 42 in the samples, the numbers above the limit of quantification (LOQ) are small and subsequently not all brain regions and doses were able to be statistically analysed. From those that were above the LOQ, there was no statistically significant effect of T30 in any brain region at any dose, compared with PBS control (1 μM: cortex p=0.8843, subcortex p=0.8138, hippocampus p=0.8494, cerebellum p=insufficient data points; 50 μM: cortex p=0.7794, subcortex p=2086, hippocampus p=0.2253, cerebellum p=insufficient data points; 100 μM: cortex p>0.9999, subcortex p=0.7484, hippocampus p=0.9975, cerebellum p=0.8069) (see FIG. 1).

Note all data for β-Amyloid 42 is shown normalised to Positive Control instead of in pg/mL. Due to the difficulties with the assay, it was decided that pg/mL would give an inaccurate quantification and therefore an unreliable representation of the data.

### Example 2—Total Tau

Referring to FIG. 2, total Tau levels were found to be significantly increased in all brain regions at both the 1 μM and 50 μM concentrations of T30 compared with PBS controls (1 μM: cortex p=0.0186, subcortex p=0.0003, hippocampus p=0.0015, cerebellum p=0.0052; 50 μM: cortex p=0.0339, subcortex p=0.0042, hippocampus p=0.0409, cerebellum p=0.0104). Total Tau levels were not significantly different in any brain region at the highest dose of T30 (100 μM), compared to PBS controls (cortex p=0.8976, subcortex p=0.9824, hippocampus p 0.6805, cerebellum p=0.5228) (FIG. 2).

Referring to FIG. 3, the percentage of Tau in dissected rat brain areas can be seen.
(i) 1 μM T30: resulted in a 50% increase of Tau in the cortex, a 90% increase of Tau in the subcortex, a 60% increase in the hippocampus and an 80% increase in the cerebellum.
(ii) 50 μM T30: resulted in a 45% increase of Tau in the cortex, a 70% increase of Tau in the subcortex, a 40% increase in the hippocampus and an 70% increase in the cerebellum.

### Example 3—T14

Referring to FIG. 4, there was no significant difference in levels of T14 at any concentration (1 μM, 50 μM, or 100 μM) of T30 compared to control (PBS) in any region of the brain analysed (cortex, subcortex, cerebellum) (1 μM: cortex p=0.3670, subcortex p=0.7354, cerebellum p=0.1273; 50 μM: cortex p=0.9917, subcortex p=0.9996, cerebellum p=0.9952; 100 μM: cortex p=0.8740, subcortex p>0.9999, cerebellum p=0.6297) (FIG. 3). It is worth noting there were limited samples remaining for the T14 analysis. No hippocampal samples were remaining to be tested.

### Example 4—Effects of AChE-Derived Peptide (T30) on Alzheimer's Disease Related Parameters (pTau, NeuN) in Sprague-Dawley Rats

Sagittal brain sections from Sprague-Dawley rats receiving either, an acute administration of T30 peptide or saline, were prepared using a cryostat as described in the methods. Every sixth section was collected starting at the midline and six sections per animal were immunostained for detection of AR (6E10), pTau (pS202/pT205), microglia (Iba1) and neurons (NeuN). Primary antibodies were combined in two co-staining sets for all animal samples. Quantitative analysis for the different markers was performed in 5 different regions of interest (ROIs) and include, cortex, hippocampus, basal forebrain, midbrain and pons/medulla.

Referring to FIG. 7, immunohistochemical staining of sections from WT Sprague Dawley rats following acute treatment with T30 peptide or saline. No intracellular pTau (yellow) is detected in hippocampus, cortex, midbrain, basal forebrain or pons/medulla. NeuN (green) is used to detect neurons and nuclei are detected with DAPI (blue). Thus, immunohistochemical results revealed that intracellular pTau (pS202/pT205) protein could not be detected in any of the stained brain sections from Sprague-Dawley rats treated with T30 peptide or saline in the cortex hippocampus, midbrain, basal forebrain or pons/medulla (see FIG. 7). Interestingly, a significant decrease in the density of NeuN positive cells was observed in the midbrain of Sprague-Dawley rats following administration of the T30 peptide compared to saline treated animals (see FIG. 7).

Referring to FIG. 8, there are shown quantification of the density of NeuN positive cells in cortex, hippocampus, midbrain, pons/medulla and basal forebrain of WT Sprague Dawley rats after treatment with T30 peptide or saline. As can be seen, no differences in the density of NeuN positive cells were observed in other brain regions including the cortex, hippocampus, basal forebrain, although a trend towards a decrease was observed in the pons/medulla region (see FIG. 8).

### Example 5—Effects of AChE-Derived Peptide (T30) on Alzheimer's Disease Related Parameters (6E10, Iba1) in Sprague-Dawley Rats

Sagittal brain sections from Sprague-Dawley were prepared and IHC was performed in the second set of co-staining for detection of Aβ and Iba1. No specific intracellular or extracellular Aβ immunoreactivity was observed in the hippocampus, cortex, midbrain, basal forebrain or pons/medulla of saline or T30 peptide treated rats (FIG. 8). Furthermore, no differences in the total number or density of Iba1 positive cells was observed in the cortex, hippocampus, cortex, midbrain, basal forebrain or pons/medulla (see FIG. 8).

### Example 6—Animal Model Behavioural Study

1) Morris Water Maze Timepoint 1

Both the MWM 6-day learning curve and the further 4-day reversal learning curve indicate that there are no significant differences in treatment groups at any day. Two-way ANOVA with repeated measures (Genotype X Day). The Probe Trial (PT) and Reversal Probe Trial (RPT) there were no significant differences between the treatment groups for their time spent in, or visits to the Target Quadrant. Two-way ANOVA (Genotype X Quadrant).

Referring to FIG. 11, however, while there were no significant differences in time spent in, or visits to the Target Platform position in the PT; a significant reduction in was found in time spent in the target platform position during the RPT for the Peptide group p=0.011. Two-way ANOVA (Genotype X Platform). Furthermore, an interaction was found between Genotype and Platform (p=0.014).

While the probe trial revealed good discrimination for the target quadrant in both treatment groups; this was less prominent in the peptide group during the reversal probe trial for visits into the target quadrant and target platform zones. This was indicated by there being no significant difference between visits to the target platform and quadrant zones and the zones previously a target in the probe trial.

2) Morris Water Maze Timepoint 2

Both the MWM 4-day learning curve and the further 4-day reversal learning curve indicate that there are no significant differences in treatment groups at any day. Two-way ANOVA with repeated measures (Genotype X Day). In the Probe Trial (PT) and Reversal Probe Trial (RPT) there were no significant differences between the treatment groups for their time spent in, or visits to the Target Quadrant. Two-way ANOVA (Genotype X Quadrant).

Referring to FIG. 12, there was a significant difference found for time spent in but not visits to the Target Platform position in the PT. The Peptide group revealed a reduction in time spent in the platform position compared with Saline controls (p=0.01). Furthermore, an interaction was found between Genotype and Platform (p<0.001). Interestingly, while a similar pattern emerged in the RPT for time spent in the platform zone, this did not achieve significance. On closer inspection this would seem to be due to one rat from the peptide group spending 2-fold longer in the platform zone during the RPT. Rats in both the PT and RPT revealed good discrimination for the target quadrant and platform zones in both treatment groups.

3) Morris Water Maze Timepoint 3

Referring to FIG. 13, no individual results from timepoint 3 were significant, however, when placed in context of timepoints 1 & 2 a trend may be seen of increasing target platform time in the saline group, while target platform time in the T30 group tends to stay the same suggesting memory impairment in the T30 group (see FIG. 14).

### Example 6—Administration of T20

Referring to FIG. 15, there is shown shows the effects, on normal rats, of a single intracerebral injection of T30 after 3 weeks, 16 weeks and 24 weeks post administration. As can be seen, the Figure includes the interim-time point histology, and shows a significant frank cell loss in a key brain region, i.e., one primarily vulnerable in Alzheimer's Disease, along with an adjacent region from the same population of vulnerable cells, also shows a significant drop.

### CONCLUSIONS

Total Tau

Total Tau levels were surprisingly found to be significantly increased in all brain regions (cortex, subcortex, hippocampus and cerebellum) for the intermediate doses (1 μM and 50 μM) of the T30 peptide, with levels returning to that of controls for the highest dose (100 μM). In all regions, the 1 μM T30 dose showed the greatest increase in Total Tau levels.

β-Amyloid 42

No significant differences were found in the levels of β-Amyloid in any region of the dissected brains (cortex, subcortex, hippocampus or cerebellum) following a single injection of T30 peptide into the basal forebrain, 2-3 weeks before rats were sacrificed. Previous research (Lin et al, 2009, J. Alzheimer's Dis, 18(4):907-18) has clearly established that increased total Tau but not β-amyloid in CSF correlates with short-term memory impairment in Alzheimer's disease. The results described herein are not inconsistent with these earlier findings of unaltered β-Amyloid levels despite significantly elevated Tau levels.

T14

There were no significant differences in the levels of T14 at any concentration of T30 in any of the samples analysed (cortex, subcortex and cerebellum) compared to controls. There were no hippocampal samples remaining to be analysed for T14 levels and there were limited numbers of other regions.

NeuN Positive Cells

The density of NeuN positive or expressing cells was significantly decreased in the midbrain, while no differences were observed in the other brain regions (cortex, hippocampus, basal forebrain or pons/medulla). NeuN levels are indicative of the number of mature neurons present.

Summary

As shown in the Figures, T30 peptide treatment induces a highly significant, dose-dependent increase in Tau in all four brain areas studied. In all cases, the highest dose (i.e. 100 M) was no different from the PBS-injected controls, which the inventors hypothesise is most likely due to a shutting down of the calcium channel when excessively stimulated (Standen, 1981, “Ca inactivation by intracellular Ca injection into Helix neurons”, Nature 293, 158-159) as seen previously with high doses of peptide applied to breast cancer cell cells (Onganer et al., 2006, “An acetylcholinesterase-derived peptide inhibits endocytic membrane activity in a human metastatic breast cancer cell line”, Biochimica et Biophysica Acta, 1760(3):415-420]) and alpha 7 transfected oocytes (Greenfield et al., 2004, “A novel peptide modulates alpha 7 nicotinic receptor responses: implications for a possible trophic-toxic mechanism within the brain”. J Neurochem 90, 325-331) as well as in brain slices (Bon et al., 2003, “Bioactivity of a peptide derived from acetylcholinesterase: electrophysiological characterization in guinea-pig hippocampus”. Eur J Neurosci 17, 1991-1995) and organotypic hippocampal neurons (Day and Greenfield 2004, “A non-cholinergic, trophic action of acetylcholinesterase on hippocampal neurones in vitro”: Molecular mechanisms. Neuroscience 111, 649-656).

However, in lower doses (less than 100 μM), where the enhanced calcium influx is viable, the T30 peptide induces activation of GSK (Garcia-Rates et al., 2016, “(I) Pharmacological profiling of a novel modulator of the α7 nicotinic receptor: Blockade of a toxic acetylcholinesterase-derived peptide increased in Alzheimer brains”. Neuropharmacology, vol 105, pp. 487-499) leading in turn to increased phosphorylation of Tau (Rankin et al., 2007, “Tau phosphorylation by GSK-3β promotes tangle-like filament morphology”. Mol Neurodegener 2: 12), in turn promoting the formation of tangles, the cardinal marker of AD (Braak and Braak 2011, “Stages of the pathologic process in Alzheimer disease: age categories from 1 to 100 years”. J Neuropathol Exp Neurol. 70(11):960-9). In other words, the inventors have surprisingly shown that low μM doses of T30 are receptor-mediated, whereas high doses are not receptor-mediated, and this was totally unexpected. The inventors believe, therefore, that the dose range of 1-99 μM T30 at which it is receptor-mediated is optimum and preferred.

FIG. 6 is a diagram showing the cascade of events resulting from the effect of T30 in a cell:

(1) T30 binds to the allosteric site of the receptor to enhance the opening of the channel for Ca²⁺ influx into the cell (Greenfield et al., 2004, “A novel peptide modulates alpha 7 nicotinic receptor responses: implications for a possible trophic-toxic mechanism within the brain”. J Neurochem 90, 325-331

(2) Calcium entry induces depolarization and opening of the voltage-dependent (L-VOCC) channel allowing still more Ca²⁺ into the cell (Dickinson et al., 2007, “Differential coupling of alpha7 and non-alpha7 nicotinic acetylcholine receptors to calcium-induced calcium release and voltage-operated calcium channels in PC12 cells”. J. Neurochem. 2007 February; 100(4):1089-96);

(3) This raised intracellular calcium induces an increase in AChE G4 release that includes T30 (Greenfield, 2013, “Discovering and targeting the basic mechanism of neurodegeneration: the role of peptides from the c-terminus of acetylcholinesterase Chemico-Biological Interactions”. 203(3):543-6);

(4) Calcium also induces upregulation of the α7 nicotinic receptor that will allow more Ca²⁺ get in the cell by providing still more targets for T30 (Bond et al., 2009, “Upregulation of alpha 7 Nicotinic Receptors by Acetylcholinesterase C-Terminal Peptides”. Plos One, 4);

(5) Calcium activates enzymes (i.e. GSK-3) that will (a) increase Tau, (b) activate y-secretase/β-secretase that will trigger cleavage of extracellular toxic Amyloid that (c) together with T30 will promote a still further toxic amount of Ca²⁺ into the cell. (Hartigan & Johnson (1999, “Transient increases in intracellular calcium result in prolonged site-selective increases in Tau phosphorylation through a glycogen synthase kinase 3beta-dependent pathway”. J Biol Chem. 23; 274(30):21395-401), Cai et al. (2012, “Roles of glycogen synthase kinase 3 in Alzheimer's disease”. Curr Alzheimer Res. 9(7):864-79.), Garcia-Rates et al (2013, “Additive Toxicity of β-Amyloid by a Novel Bioactive Peptide In Vitro: Possible Implications for Alzheimer's Disease”. PLoS ONE 8(2):e54864.)).

1. A method of providing an animal model for a neurodegenerative disease, the method comprising introducing, into the brain of a non-human animal, a peptide comprising or consisting of the amino acid sequence represented as SEQ ID NO: 3, or an active variant of fragment thereof, wherein the peptide causes an increase in Tau protein in one or more sites in the animal's brain.

2. A method according to claim 1, wherein the method comprises introducing the peptide or variant or fragment thereof into the brain of a wild-type non-human animal.

3. A method according to claim 1, wherein administration of the peptide or variant or fragment thereof to the non-human animal causes an increase in Tau protein in one or more sites in the animal's brain selected from a group consisting of: the cortex; subcortex; hippocampus; cerebellum; basal forebrain; and pons/medulla region, optionally wherein administration of the peptide or variant or fragment thereof causes an increase in Tau protein in at least one, two, three, four, five or all six sites in the animal's brain selected from a group consisting of: the cortex; subcortex; hippocampus; cerebellum; basal forebrain; and pons/medulla region.

4. A method according to claim 1, wherein administration of the peptide, or variant or fragment thereof causes an increase in Tau protein in the one or more sites in the animal's brain by at least 1%, 3%, 5%, 10% or 20% compared to an untreated control.

5. A method according to claim 1, wherein administration of the peptide, or variant or fragment thereof causes an increase in Tau protein in the one or more sites in the animal's brain by at least 30%, 40% or 50% compared to an untreated control.

6. A method according to claim 1, wherein administration of the peptide or variant or fragment thereof to the non-human animal causes a decrease in neurons in one or more sites in the animal's brain selected from a group consisting of: the cortex; subcortex; hippocampus; cerebellum; basal forebrain; and pons/medulla region, optionally wherein administration of the peptide or variant or fragment thereof causes an increase in Tau protein in at least one, two, three, four, five or all six sites in the animal's brain selected from a group consisting of: the cortex; subcortex; hippocampus; cerebellum; basal forebrain; and pons/medulla region.

7. A method according to claim 1, wherein the peptide or variant or fragment thereof, comprises or consists of at least 15, 16, 17, 18 or 19 amino acids of the sequence represented as SEQ ID NO: 3, or wherein the variant or fragment of the peptide administered to the brain of the non-human animal comprises or consists of at least 20, 21, 22, 23 or 24 amino acids of the sequence represented as SEQ ID NO: 3.

8. A method according to claim 1, wherein the peptide or variant or fragment thereof comprises or consists of at least 25, 26, 27, 28 or 29 amino acids of the sequence represented as SEQ ID NO: 3.

9. A method according to claim 1, wherein the peptide or variant or fragment thereof comprises or consists of at least 15, 20, 25 or 30 amino acid residues and has at least 90% or 95% sequence identity with SEQ ID No: 3.

10. A method according to claim 1, wherein the concentration of the peptide or variant or fragment thereof being administered to the animal is less than 1 mM, or less than 750 μM, or less than 500 μM, or less than 400 μM, or less than 300 μM, or less than 200 μM, or less than 100 μM, or less than 75 μM, or less than 60 μM.

11. A method according to claim 1, wherein the concentration of the peptide or variant or fragment thereof is less than 50 μM, or less than 40 μM, or less than 30 μM, or less than 20 μM, or less than 10 μM, or less than 5 μM, or less than 3 μM.

12. A method according to claim 1, wherein the concentration of the peptide or variant or fragment thereof being administered is more than 0.01 μM, or more than 0.1 μM, or more than 1 μM, or more than 3 μM, or more than 5 μM, or more than 10 μM, or more than 20 μM.

13. A method according to claim 1, wherein the concentration of the peptide or variant or fragment thereof being administered is more than 30 μM, or more than 40 μM, or more than 50 μM, or more than 60 μM, or more than 70 μM, or more than 80 μM, or more than 90 μM.

14. A method according to claim 1, wherein the concentration of the peptide or variant or fragment thereof being administered is between 0.01 μM and 1000 μM, or between 0.1 μM and 500 μM, or between 1 μM and 100 μM, or between 1 μM and 90 μM.

15. A method according to claim 1, wherein the concentration of the peptide or variant or fragment thereof is between 0.1 μM and 80 μM, or between 0.1 μM and 70 μM, or between 0.1 μM and 60 μM, or between 0.1 μM and 50 μM, or between 0.1 μM and 40 μM, or between 0.1 μM and 30 μM, or between 0.1 μM and 20 μM, or between 0.1 μM and 10 μM.

16. A method according to claim 1, wherein the concentration of the peptide or variant or fragment thereof is between 10 μM and 80 μM, or between 20 μM and 80 μM, or between 30 μM and 70 μM, or between 40 μM and 60 μM.

17. A method according to claim 1, wherein the concentration of the peptide or variant or fragment thereof is 0.1-99 μM.

18. A method according to claim 1, wherein the peptide or variant or fragment thereof is introduced into the basal forebrain region of the brain.

19. A method according to claim 1, wherein the peptide or variant or fragment thereof is introduced into: (i) the medial septum/diagonal band of Broca (SID13) region of the brain; (ii) the cortical cholinergic system; and/or (iii) the nucleus basalis magnocellularis (NBM).

20. A method according to claim 1, wherein the non-human animal is a mammal.

21. A method according to claim 1, wherein the animal is a primate, optionally a monkey.

22. A method according to claim 1, wherein the non-human animal is a rodent, optionally a mouse or a rat.

23. A method according to claim 1, wherein the peptide or a variant or fragment thereof contributes to, or causes, neurodegeneration.

24. A method according to claim 1, wherein the peptide or variant or fragment thereof administered to the animal model causes cellular degeneration and thereby impairment of a testable brain function, wherein impairment of the same brain function in a human is indicative of a neurological disorder.

25. A method according to claim 1, wherein the method or model is used to investigate any neurodegenerative disease characterised by tauopathy.

26. A method according to claim 1, wherein the neurodegenerative disease is selected from a group consisting of: Alzheimer's disease; Parkinson's disease; Motor Neurone disease; Spinocerebellar type 1, type 2, and type 3; Amyotrophic Lateral Sclerosis (ALS); Lewy-body dementia; and Frontotemporal Dementia.

27. A method according to claim 1, wherein neurodegenerative disease is Alzheimer's Disease, Parkinson's Disease or Motor Neuron Disease.

28. A method according to claim 24, wherein the testable brain function, the impairment of which is tested, is cognitive function or attentional deficit.

29. A method according to claim 1, wherein the method comprises testing the animal model for the impairment of an appropriate brain function, optionally by providing the animal with an attentional task to test an attentional impairment.

30. A method according to claim 1, wherein the method further comprises administering prior to, simultaneously or after the peptide, or variant or fragment thereof, a test agent and determining whether the agent inhibits, prevents or increases impairment of a testable brain function and/or inhibits, prevents or increases cellular damage in the brain.

31. A method according to claim 1, wherein cellular damage comprises neurodegeneration, optionally wherein the damage is monitored or assessed by measuring one or more of:
(i) the inhibition of activity in neuronal populations (i.e. assemblies);
(ii) calcium levels;
(iii) acetylcholinesterase activity levels;
(iv) expression of alpha-7 nicotinic receptors in cell membranes; and
(v) cell density and/or loss or reduction of NeuN-expressing cells (related to neuronal death) in specific areas.

32. An animal model for a neurodegenerative disease, which is a non-human animal treated with a peptide comprising or consisting of the amino acid sequence represented as SEQ ID NO: 3, or an active variant of fragment thereof.

33. An animal model prepared using the method according to claim 1.

34. Use of the animal model according to claim 32 to: (i) examine neurodegenerative or neuroregenerative processes; (ii) test pharmacological compounds which may modulate neurodegenerative or neuroregenerative processes; or (iii) screen neurodegenerating or neuroregenerating drugs.

35. A method of identifying a candidate agent, for use in the treatment, prevention or amelioration of neurodegenerative disorder, the method comprising:
administering a candidate agent to the animal model according to claim 32; and
determining if the candidate agent inhibits, prevents or increases impairment of a testable brain function and/or causes improvement or deterioration of cellular damage in the brain,
wherein inhibition or prevention of impairment of the testable brain function, or improvement of cellular damage in the brain indicates that the test agent is a candidate for the treatment, prevention or amelioration of neurodegenerative disorder, whereas increasing impairment of the testable brain function or deteriorating cellular damage in the brain indicates that the agent is not a candidate for the treatment, prevention or amelioration of neurodegenerative disorder.

36. A method according claim 35, wherein the testable brain function is a cognitive function or an attentional deficit, optionally wherein the method comprises testing the animal model for impairment or a cognitive function or an attentional deficit.

37. A method of testing a test agent for biological activity in a neurodegenerative disease, wherein the method comprises administering the test agent to the animal model according to claim 32, and assessing the animal having a brain lesion for any change, either improvement or deterioration, associated with the brain lesion.

38. A method according to claim 37, wherein the assessment comprises determining whether the agent inhibits, prevents or increases impairment of an appropriate testable brain function, optionally a cognitive function such as attention or memory, and/or determining whether there is any improvement or deterioration in cellular damage at the relevant site(s) in the brain.

39. A method according to claim 37, wherein the test agent is a drug compound.


SEQUENCE LISTING (5 sequences)

SEQ ID NO: 1 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 2 - protein, 42 aa, Homo sapiens [sequence omitted]

SEQ ID NO: 3 - protein, 30 aa, Homo sapiens
KAEFHRWSSYMVHWKNQFDHYSKQDRCSDL

SEQ ID NO: 4 - protein, 14 aa, Homo sapiens
AEFHRWSSYMVHWK

SEQ ID NO: 5 - protein, 15 aa, Homo sapiens
NQFDHYSKQDRCSDL